(19) United States
(12) Patent Application Publication
(10) Pub. No.: US 2011/0160274 A1
(43) Pub. Date: Jun. 30, 2011

(54) FENOFIBRATE FORMULATION WITH ENHANCED ORAL BIOAVAILABILITY

(75) Inventors: Rajesh Jain, New Delhi (IN); Sarabjit Singh, Maharashtra (IN); Naveen Jain, Maharashtra (IN); Shivanand Puthli, Maharashtra (IN)

(73) Assignee: PANACEA BIOTEC LIMITED, Navi Mumbai Maharashtra (IN)

(21) Appl. No.: 13/002,372

(22) PCT Filed: Jun. 26, 2009
(86) PCT No.: PCT/IN2009/000365

§ 371 Date: Jan. 26, 2011

(30) Foreign Application Priority Data
Jul. 3, 2008 (IN) 1384/MUM/2008

(51) Int. Cl. A61K 31/40; A61K 31/216; A61P 3/10; A61P 9/00

(52) U.S. Cl. 514/423; 514/543

(57) ABSTRACT

The present invention provides a formulation of fenofibrate with enhanced oral bioavailability, simplicity of design and manufacture and absence of food effect. The formulation comprises fenofibrate dissolved in a lipophilic surfactant, with a hydrophilic surfactant optionally added. The formulation can be effectively used in the management and treatment of conditions such as hypertriglyceridemia, hypercholesterolemia and mixed dyslipidemia, and can also be effective at lower doses as compared to commercially available products. The invention additionally relates to the process of manufacture of the formulation and to dosage forms comprising the same.

### FIELD OF THE INVENTION

The present invention relates to a novel formulation of fenofibrate with enhanced oral bioavailability, to a process for its manufacture and to dosage forms comprising the formulation.

### BACKGROUND OF THE INVENTION

Fenofibrate or 2-[4-(4-chlorobenzoyl)phenoxy]-2-methyl-propanoic acid, 1-methylethyl ester belongs to a class of lipid regulating agents known as fibrates, which are useful in reducing elevated serum triglyceride levels in hypertriglyceridemic patients and cholesterol and LDL-C levels in hypercholesterolemic and mixed dyslipidemic patients.

Physically, fenofibrate is a white solid, virtually insoluble in water. On oral administration, it is absorbed and metabolized to the active substance fenofibric acid, which has a plasma elimination half life of about 20 hours. It is well known that poor aqueous solubility limits the dissolution and hence absorption of fenofibrate from the gastrointestinal tract. Despite its poor solubility, it is reported to be well absorbed when dosed in the “fed state” and less so in the “fasted state”. Various attempts have been made to improve the solubility and bioavailability of fenofibrate, such as use of surface active agents and surface stabilizers with fenofibrate and by elaborate manufacturing processes such as micronisation and spray-drying to reduce the effective average particle size of fenofibrate. Solubility enhancement has also been attempted by dissolving it in agents such as organic solvents, oily materials and triglycerides. Different hydrophilic and hydrophobic agents have been evaluated for the purpose.

For example, PCT Application No. 2006107411 discloses fibrate compositions with fibrates in intimate association with PEG and Poloxamer 407. U.S. Pat. No. 5,545,628 claims fenofibrate with polyglycolized glycerides. U.S. Pat. No. 6,814,977 discloses fenofibrate dissolved in a medium chain glycerol ester of fatty acid. U.S. Pat. No. 6,719,999 discloses fenofibrate dissolved in glycerin, propylene glycol, or dimethylisosorbide and U.S. Pat. No. 5,827,536 discloses fenofibrate dissolved in diethyleneglycol monoethyl ether.

A U.S. Pat. No. 6,294,192 discloses a capsule dosage form for any hydrophobic therapeutic agent having a carrier system which comprises of at least one hydrophilic surfactant and at least one hydrophobic surfactant having an HLB value of less than about 10. The carrier system contains the surfactants in such amounts that on dilution with water, a clear aqueous dispersion is obtained. For this purpose, the amount of hydrophilic surfactant has to be considerably higher than the hydrophobic surfactant. As mentioned in its specification, the hydrophobic surfactant is less than about 200% by weight of the hydrophilic surfactant, and preferably is about 10 to 60% by weight of the hydrophilic surfactant.

One of the recent products using the lowest effective doses of fenofibrate so far is a tablet composition launched by Lifecycle Pharma in United States under the brand name Fenoglide®. It is available in two doses of 40 mg and 120 mg. Lifecycle Pharma's United States patent application No. 20070026062 describes a solid dosage form comprising a solid dispersion or solid solution of a fibrate. The composition contains the fibrate in a solid form. It is prepared by techniques such as spray drying, controlled agglomeration, freeze drying, coating on carrier particles and other solvent removal processes, wherein the vehicle is generally a solid which has to be brought in a liquid form by processes such as melting.

Thus, numerous attempts have been made in the direction of solubility enhancement of fenofibrate, although few have translated in satisfactory products. It would be advantageous to develop a formulation of fenofibrate which improves its solubility, demonstrates superior bioavailability over the commercially available products and at the same time involves the use of least number of excipients and is simple to manufacture. It would also be advantageous to have a formulation of fenofibrate which is similarly effective in both fed and fasted state of the subject and hence which can be administered without regards to meals.

In addition, fenofibrate activity has also been investigated in combination with other cardiovascular agents commonly for the prophylaxis and treatment of conditions such as hyperlipemia, atherosclerosis, hypercholesterolemia and related disorders. For example, combination of fenofibrate with antilipidemic agents such as statins and sterol absorption inhibitors (e.g. Ezetimibe); cholesteryl ester transfer protein inhibitors such as Torcetrapib; benzoquinones such as coenzyme Q10 etc. have been disclosed in the literature. Combination of fenofibrate with antilipidemic agents like the HMG-CoA reductase inhibitors (statins) can produce a synergistic effect in lowering cholesterol levels and the combination can be used to treat conditions such as dyslipidemia, hyperlipidemia, hypercholesterolemia and other related conditions. Hence, it would be advantageous that the formulation of the present invention can further comprise of additional active agents, such as antilipidemic agents like statins, and can provide adequate delivery of the agents to produce therapeutically effective levels in the body.

### BRIEF DESCRIPTION OF THE INVENTION

The inventors of the present invention have found a formulation which surprisingly, while being extremely simple in manufacture and design, has demonstrated unexpectedly enhanced bioavailability for fenofibrate. It has also shown promising results with regard to reducing or even eliminating the food effects of fenofibrate. Moreover, the unexpectedly superior bioavailability of the present formulation may translate into the formulation being effective at a lower dose, thereby also improving on the side effect profile.

Accordingly, the present invention provides a formulation comprising fenofibrate dissolved in a lipophilic surfactant. Preferably, it also comprises of another surfactant which is hydrophilic in nature and the weight ratio of lipophilic and hydrophilic surfactants is between 1:2 to 2:1.

An aspect of the invention also relates to a process for manufacture of the fenofibrate formulation with enhanced oral bioavailability, comprising dissolving fenofibrate in a lipophilic surfactant, optionally adding the hydrophilic surfactant and mixing to obtain a clear or slight hazy solution.

Certain embodiments relate to the present formulation with additional active agents included, such as nicotinic acid, HMG-CoA reductase inhibitors, sterol absorption inhibitors and bile acid sequestrants and to processes for their preparation.

Finally, an aspect of the invention relates to dosage forms incorporating the present formulation, which can preferably be capsule dosage forms.

### DESCRIPTION OF THE DRAWINGS

FIG. 1 shows the graphical comparison of fenofibrate plasma concentration time profiles of the Test formulation A (from Example 1A) vs. Reference formulation B (commercially available product Tricor®) in Wistar rats.

FIG. 2 shows the graphical comparison of plasma concentration time profiles of fenofibrate from Example 4 in fed vs. fasted rats which were administered the Test formulation A.

FIG. 3 depicts the graphical comparison of fenofibric acid levels in plasma of human volunteers who were administered the Test formulation of Example 5 (with 130 mg fenofibrate) in fed and fasted states. The comparison indicates the absence of a significant food effect.

FIG. 4 depicts the graphical comparison of fenofibric acid levels in plasma of human volunteers who were administered the Reference formulation Storfib® (with 145 mg fenofibrate) in fed and fasted states. The comparison indicates the presence of a significant food effect.

### DETAILED DESCRIPTION OF THE INVENTION

As summarized hereinabove, the present invention provides a formulation comprising fenofibrate dissolved in a lipophilic surfactant. Preferably, it also comprises of another surfactant which is hydrophilic in nature. Optimal results have been obtained when both types of surfactants are present and the weight ratio of the lipophilic and hydrophilic surfactants has been from 1:2 to 2:1. Without wishing to be bound by theory, it is believed that the lipophilic surfactant maintains the fenofibrate in a dissolved and easily absorbable state, while the hydrophilic surfactant ensures sufficient availability of fenofibrate at the site of absorption. The formulation may further include such other auxiliary excipients as may be required for optimal manufacture and use.

The lipophilic surfactants which have demonstrated unexpectedly good results belong to the class of esters of propylene glycol. The esters are generally of fatty acids such as laurate, caprylate, stearate, ricinoleate, oleate etc. and include both mono- and di-esters. Some non-limiting examples include propylene glycol monolaurate, propylene glycol monocaprylate, propylene glycol dicaprylate/dicaprate, propylene glycol monooleate, propylene glycol ricinoleate, propylene glycol distearate, propylene glycol myristate, propylene glycol monostearate, propylene glycol isostearate etc. They are preferably used in the amounts of 20% w/w to 80% w/w of the formulation. A particularly preferred lipophilic surfactant is propylene glycol monocaprylate, available from Gattefosse under the tradename of Capryol 90® or Capryol PGMC®.

In an embodiment of the invention, hydrophilic surfactants are also used. They belong to a unique class of nonionic surfactants known as polyoxyethylene sorbitan fatty acid esters. This class includes a series of partial fatty acid esters of sorbitol and its anhydrides copolymerized with different moles of ethylene oxide. The generic name for these compounds is ‘polysorbates’. Preferred polysorbates are those containing 20 units of oxyethylene, such as the Polysorbate 80. Polysorbate 80, also known as Tween 80, is a viscous, water-soluble ester of polyoxylated sorbitan and oleic acid. Satisfactory results are obtained when the polysorbates are included in the formulation in the range of 80% w/w to 20% w/w.

In an alternative embodiment of the invention, another class of hydrophilic surfactants is used. They are polymeric surfactants known as polyoxyethylene-polyoxypropylene block copolymers. This class includes various agents with hydrophilic and hydrophobic moieties present in well defined ratios and positions, providing compounds with a wide range of hydrophilic—hydrophobic characteristics. The generic name for these compounds is ‘poloxamers’. Preferred poloxamers for the present invention are the hydrophilic poloxamers such as poloxamer 108, 188, 217, 238, 288, 338 and 407. Satisfactory results are obtained when they are included in the formulation in the range of 80% w/w to 20% w/w.

Other optional excipients may be included in the present formulation to modify its characteristics to an optimal level. They include antioxidants such as tocopherol, ascorbyl palmitate, ascorbic acid, butylated hydroxytoluene, butylated hydroxyanisole, propyl gallate, etc.; pH stabilizers such as citric acid, tartaric acid, fumaric acid, acetic acid, glycine, arginine, lysine, potassium hydrogen phosphate; other suitable buffers, preservatives, thickeners, colors, flavours etc. Overall, the formulation is extremely simple in design and utilizes least number of excipients.

Fenofibrate, used as the active ingredient in the formulation does not require to be specially processed in any way, and can be either micronised or unmicronised. Incidentally, it also encompasses a derivative (e.g. an ester), a salt, a prodrug or the active moiety itself. The amount of fenofibrate contained in the formulation is any therapeutically effective amount, and generally ranges from about 30 mg to about 200 mg. Preferably, the specific doses could be 40 mg, 43 mg, 48 mg, 50 mg, 54 mg, 67 mg, 100 mg, 107 gm, 130 mg, 134 mg, 145 mg, 150 mg, 160 mg and 200 mg. In vivo studies and clinical trials, included hereinafter, have shown that in comparison with commercially available fenofibrate products, the present formulation demonstrates unexpectedly enhanced bioavailability; thereby exhibiting sufficient therapeutic effect in smaller doses as compared to the commercially available oral products. Hence it is strongly believed that a lower daily dose of the active ingredient can be administered to the patient eg. a 130 mg, 120 mg or even a 100 mg dose of the fenofibrate may be administered through the present formulation. It is similarly contemplated that the reduced dose will lead to a reduction of side effects generally observed with the use of fenofibrate, such as nausea, muscle pain, back-pain and body weakness. Furthermore, the in vivo studies and clinical trials have clearly demonstrated that the formulation of the invention significantly reduces or even eliminates food effect; i.e. the absorption of fenofibrate is relatively independent of whether the patient takes the formulation on a full or empty stomach. Accordingly the patient has the choice of taking the formulation once daily at any convenient time, without regard to his meals. This is in contrast to some of the commercially available preparations which exhibit significant food effect.

In certain other embodiments, the formulation of the invention may also comprise of one or more additional active agents such as antidiabetics like DPP-IV inhibitors, alpha glucosidase inhibitors e.g. voglibose, acarbose, biguanides e.g. metformin, PPAR agonists e.g. rosiglitazone, pioglitazone; calcium antagonists like verapamil, amlodipine, felodipine; ACE inhibitors like enalapril, ramipril, lisinopril, quinapril; fats and vitamins like Vitamin E, nicotinic acid, vitamin B, folic acid, betaine, omega-3 fatty acids, Coenzyme Q10; NSAIDs, platelet aggregation inhibitors like aspirin and clopidogrel; bisphosphonates etc. Preferably, the additional active agents are other antilipidemic agents like nicotinic acid; HMG-CoA reductase inhibitors (commonly known as statins) such as atorvastatin, rosuvastatin, pravastatin, fluvastatin, lovastatin, simvastatin, mevastatin, itavastatin, cerivastatin; sterol absorption inhibitors such as ezetimibe; bile acid sequestrants such as cholestyramine, colestipol, DEAE-Sephadex etc. Certain embodiments may also include triple combinations such as fenofibrate with statins and Ezetimibe, statins and nicotinic acid, Ezetimibe and bile acid sequestrants, Nicotinic acid derivative and Sterol absorption inhibitor, α-glucosidase inhibitor and statins and so on.

HMG-CoA reductase inhibitors and fenofibrate work by complimentary mechanisms of action to produce an overall reduction in the plasma cholesterol levels. Fibrates reduce the levels of serum triglycerides and low density lipoprotein binding cholesterol by inhibiting the synthesis or secretion of triglycerides in the liver, while increasing the high density lipoprotein binding cholesterol. HMG-CoA reductase inhibitors suppress the synthesis of cholesterol by inhibiting hydroxymethylglutaryl CoA (HMG-CoA) reductase, which is a rate determining enzyme in the biosynthetic pathway of the cholesterol. For example, it has been well reported that a combination of atorvastatin and fenofibrate results in greater improvements in multiple lipid parameters in patients with dyslipidaemia than either drug alone. To incorporate the therapeutic benefits of this synergistic combination, an embodiment of the invention relates to a formulation comprising fenofibrate dissolved in a lipophilic surfactant and containing atorvastatin as an additional active agent. The formulation comprises atorvastatin in dose range of 2 mg to 100 mg although preferably it is from 5 to 80 mg, and more preferably from 5 to 40 mg. Fenofibrate can be included in the range of from 30 mg to 200 mg, preferably from 40 mg to 160 mg, Moreover, since the formulation exerts sufficient effects in a smaller dose as compared with the dose of each of the active ingredient alone, as known in the literature and as demonstrated in the clinical trials, it is possible to incorporate reduced doses of fenofibrate in the formulation of the invention. Specifically, a reduced dose of fenofibrate such as 145 mg, 130 mg, 120 mg, or even 100 mg has been contemplated. The formulation may also further comprise of optional ingredients such as antioxidants like tocopherol, ascorbyl palmitate, ascorbic acid, butylated hydroxytoluene, butylated hydroxyanisole, propyl gallate, etc.; pH stabilizers such as citric acid, tartaric acid, fumaric acid, acetic acid, glycine, arginine, lysine, potassium hydrogen phosphate, Calcium carbonate, Calcium hydroxide, Calcium citrate, Magnesium citrate, Zinc carbonate, Magnesium carbonate, Magnesium hydroxide, Magnesium trisilicate, Sodium carbonate, Tromethamine, Sodium bicarbonate, Trimagnesium phosphate octa-hydrate, d1-alpha glycerol phosphate magnesium hydrate, Potassium hydroxide, Tromethamine, Amberlite, Propylene carbonate, Carbopol; other suitable buffers, preservatives, thickeners, colors, flavours etc.

Incidentally, fenofibrate and the additional active agents (such as statin compounds) of the invention also encompass a derivative of each compound (e.g. an ester), a salt, a prodrug or the active moiety itself.

An aspect of the invention also relates to the process for manufacture of the fenofibrate formulation. The process is extremely simple and involves dissolving fenofibrate in the lipophilic surfactant, with the help of slight heat if necessary, optionally adding a hydrophilic surfactant to it and adequately mixing to get a clear or slight hazy solution. The solution may be further diluted with additional liquids or may be thickened and/or stabilized with various pharmaceutical excipients to vary its characteristics. Exemplarily, fenofibrate particles, either micronised or unmicronised, are dissolved in a lipophilic surfactant such as a propylene glycol ester, for example propylene glycol monocaprylate. These surfactants are generally semi-solid or viscous liquid in nature and can be used to dissolve fenofibrate by stirring or by applying slight heat, if necessary. Heating up to a temperature of about 45 to 55° C. generally produces satisfactory results. A hydrophilic surfactant, such as a poloxamer or a polysorbate is optionally added to the lipophilic surfactant and adequately stirred. By way of illustration, a formulation may be prepared wherein about 0.1% w/w to 50% w/w of fenofibrate is dissolved in about 20% w/w to 80% w/w of lipophilic surfactant. About 80% w/w to 20% w/w of hydrophilic surfactant is added to the solution and mixed till a clear or slight hazy solution is obtained. Other optional excipients, such as antioxidants, pH stabilizers, buffers, preservatives, thickeners, colors, flavours, may be added, if required. The formulation is then incorporated into a suitable dosage form and packaged.

Accordingly, a further aspect of the invention relates to a dosage form comprising the present formulation. The dosage form could be any of those known in the art and suitable for including the formulation of the invention. It may be a liquid preparation or a semi-solid preparation in a container, or a lozenge etc. More preferred are the capsule dosage forms. Soft gelatin capsules, also known as Softgels, are hermetically sealed, one-piece capsules with a liquid or semisolid fill. Especially preferred are these capsules, which can be filled with the formulation and sealed.

Thus an aspect of the invention relates to a process for manufacture of a fenofibrate formulation comprising the following steps:
a. dissolving fenofibrate and optionally a hydrophilic surfactant together or sequentially, in a lipophilic surfactant,
b. stirring the mixture well and applying heat if necessary, to produce a clear or slight hazy solution,
c. optionally adding other excipients to the mixture of step b,
d. incorporating the mixture into a dosage form.

In an embodiment, the process comprises of the following steps:
a. dissolving fenofibrate and a hydrophilic surfactant together or sequentially, in a lipophilic surfactant,
b. stirring the mixture well and applying heat if necessary, to produce a clear or slight hazy solution,
c. optionally adding other excipients included from the group of antioxidants, pH stabilizers, buffers, preservatives, thickeners, colors and flavours to the mixture of step b,
d. incorporating the mixture into a capsule dosage form.

In another embodiment, the process comprises of the following steps:
a. dissolving fenofibrate and a hydrophilic surfactant selected from the group of Poloxamers and Polysorbates together or sequentially, in an ester of propylene glycol,
b. stirring the mixture well and applying heat if necessary, to produce a clear or slight hazy solution,
c. optionally adding other excipients included from the group of antioxidants, pH stabilizers, buffers, preservatives, thickeners, colors and flavours to the mixture of step b,
d. incorporating the mixture into a soft gelatin capsule dosage form.

Further, an aspect of the invention also relates to a process for manufacture of a fenofibrate formulation comprising one or more additional active agents, the process comprising the following steps:
a. dissolving fenofibrate, one or more additional active agent and optionally a hydrophilic surfactant together or sequentially, in a lipophilic surfactant,
b. stirring the mixture well and applying heat if necessary, to produce a clear or slight hazy solution,
c. optionally adding other excipients to the mixture of step b,
d. incorporating the formulation into a dosage form.

In an aspect, the invention relates to the incorporation of an antilipidemic agent, preferably an HMG coA reductase inhibitor such as atorvastatin, in the above process. Thus it relates to a process for manufacture of a formulation comprising fenofibrate and atorvastatin, the process comprising the following steps:
a. dissolving fenofibrate, atorvastatin and a hydrophilic surfactant together or sequentially, in a lipophilic surfactant,
b. stirring the mixture well and applying heat if necessary, to produce a clear or slight hazy solution.
c. optionally adding other excipients included from the group of antioxidants, pH stabilizers, buffers, preservatives, thickeners, colors and flavours to the mixture of step b,
d. incorporating the formulation into a capsule dosage form.

In a more specific embodiment, 30 mg to 200 mg of fenofibrate and 5 mg to 80 mg of atorvastatin are dissolved in a fatty acid ester of propylene glycol and a hydrophilic surfactant selected from the group of poloxamers and polysorbates is added to it.

The invention, as described hereinabove, will be understood more clearly from the following non-limiting representative examples:

### Example 1

| | | Qty (mg/capsule) | Qty (mg/capsule) |
|---|---|---|---|
| Sr. No. | Ingredient | 1A | 1B |
| 1 | Fenofibrate | 100.0 | 130.0 |
| 2 | Propylene glycol | 337.5 | 671.0 |
| | monocaprylate | | |
| 3 | Polysorbate 80 | 437.5 | 439.0 |

Fenofibrate was dissolved in Propylene glycol monocaprylate with the use of slight heat. Polysorbate 80 was added and mixed adequately. The solution was filled into soft gelatin capsules.

### Example 2

| Sr. No. | Ingredient | Qty (% w/w) |
|---|---|---|
| 1 | Fenofibrate | 11 |
| 2 | Propylene glycol | 78.8 |
| | monocaprylate | |
| 3 | Poloxamer 407 | 10.2 |

Fenofibrate was dissolved in Propylene glycol monocaprylate with the use of slight heat. Poloxamer 407 was added and mixed adequately. A hazy solution was obtained. The solution was filled into soft gelatin capsules.

### Example 3

In vivo studies to demonstrate the bioavailability of the present formulation relative to the bioavailability of the commercially available reference fenofibrate formulation, i.e. Tricor®, were carried out.

Wistar rats (n=6) (fasted) were treated orally with 2 different formulations of fenofibrate. Formulation A was the fenofibrate formulation of the invention as given in Example 1A, at the dose of 62 mg/kg body weight of rat. Formulation B was the commercial product Tricor® administered at the dose of 90 mg/kg body weight of rat.

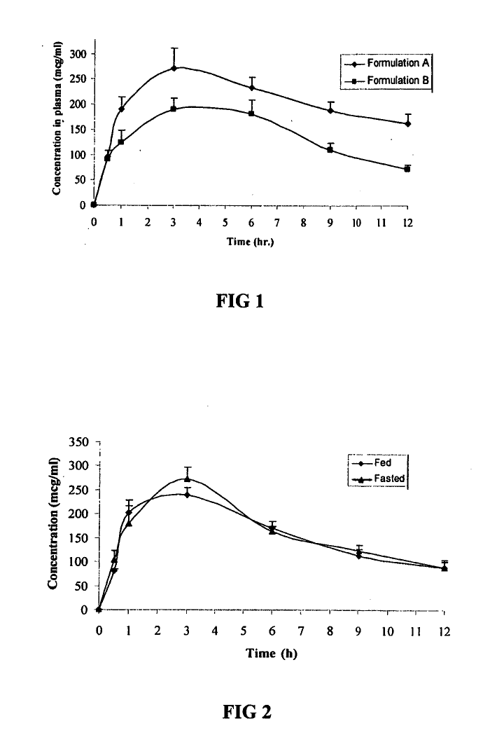

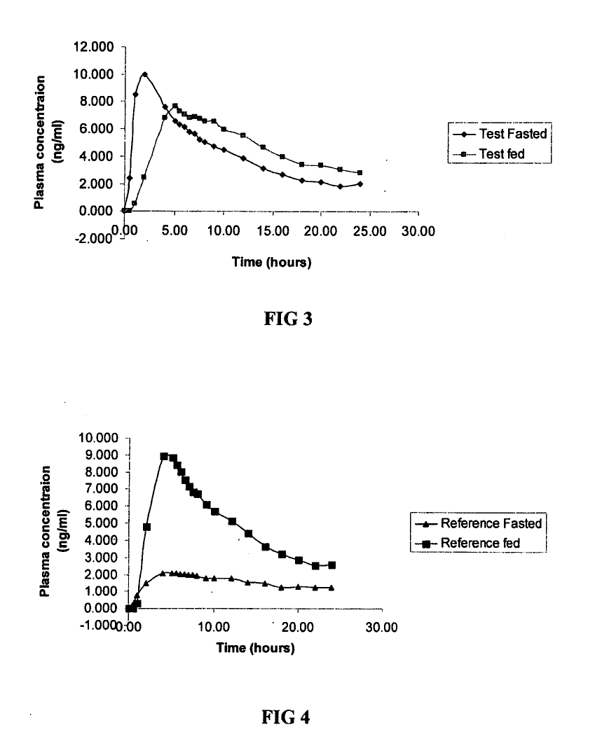

Blood samples were collected from the retro-orbital sinus at regular intervals of 0, 0.5, 1, 3, 6, 9 and 12 hours, and the plasma was submitted for analysis of fenofibric acid by LC-MS/MS. FIG. 1 represents the resulting data in graphical form.

The results, indicated as mean±Sem, n=6, are as follows:

| | | Cₘₐₓ | AUC_{(0-t last)} |
|---|---|---|---|
| Formulation | Tₘₐₓ (hr) | (μg/ml) | (μg-hr/ml) |
| A | 3 | 271.81 ± 40.02 | 2473.4 ± 122.5 |
| B | 3 | 189.08 ± 23.87 | 1648.7 ± 85.1 |

The fenofibrate formulation demonstrated a relative bioavailability of 150% as compared to Tricor® even at a much reduced dose of 62 mg/kg body weight of rat as compared to the dose of 90 mg/kg body weight of rat for Tricor®. The results thus indicate a superior fenofibrate formulation of the invention with surprisingly enhanced oral bioavailability.

### Example 4

Further in vivo studies were designed to evaluate the effect of food, if any, on the pharmacokinetics of fenofibrate administered through the formulation of the present invention. Fed and overnight fasted (12 h) rats were treated orally with the formulation A of the invention. Blood samples (400 μL) were collected from retro-orbital sinus at regular intervals as mentioned above, and the plasma was analyzed for fenofibric acid by LC-MS/MS. FIG. 2 represents the data in graphical form.

The results, indicated as mean±Sem, n=5-6, are as follows:

| | AUC_{(0-t last)} | | Cₘₐₓ |
|---|---|---|---|
| Formulation | μg-hr/ml | Tₘₐₓ (hr) | μg/ml |
| A (Fed) | 1874.9 ± 93.9 | 3.0 | 239 ± 15.5 |
| A (Fasted) | 1952.6 ± 98.5 | 3.0 | 273.4 ± 24.6 |

The results indicate comparable AUC, Cₘₐₓ and Tₘₐₓ values. There is no significant difference in the plasma profiles of fenofibric acid after administration of fenofibrate under fed and fasted states, indicating an absence of food effect.

### Example 5

A Formulation comprising fenofibrate 130 mg and an additional active agent Atorvastatin 10 mg was also evaluated for bioavailability and food effect. Reference formulation was the commercially available product Storfib® (manufactured by Ranbaxy Laboratories Ltd., India, containing Fenofibrate 145 mg and Atorvastatin 10 mg.). Open label, balanced, randomized, two treatment, two-sequence two period, single dose, crossover comparative bioavailability study in healthy adult human subjects was carried out under fed and fasted conditions. Total 12+2 healthy male volunteers who met all the inclusion criteria were recruited for the study.

Fed and overnight fasted (for fed and fasted studies respectively) male volunteers were administered either of the Test or Reference formulation and a total of 21 blood samples were collected from the subjects during each period. Venous blood samples (10 ml each) were withdrawn pre-dose and 5 ml each at 0.50, 1.0, 2.0, 4.0, 5.0, 5.5, 6.0, 6.5, 7.0, 7.5, 8.0, 9.0, 10.0, 12.0, 14.0, 160, 18.0, 20.0, 22.0 and 24 hours after dosing. A washout of at least 7 days was kept from the completion of dosing between two consecutive periods. Pharmacokinetic parameters evaluated were Cmax, AUC(0-t), AUC(0-inf). Figures III and IV represent the resulting data in graphical form.

The results for Fenofibrate, for both the fed and fasted studies, are as indicated in the Table below:

| | Fed study | | | | | Fasted study | | | | |
|---|---|---|---|---|---|---|---|---|---|---|
| PK | | | Ratio | 90% CI | | | | Ratio | 90% CI | |
| Parameter | Reference | Test | (T/R) | Lower | Upper | Reference | Test | (T/R) | Lower | Upper |
| LCmax | 9.3753 | 8.4777 | 90.43 | 82.40 | 99.23 | 2.2754 | 10.2113 | 448.76 | 374.66 | 537.53 |
| LAUCt | 109.7979 | 106.6390 | 97.12 | 93.24 | 101.17 | 36.0641 | 100.1664 | 277.75 | 257.02 | 300.14 |
| LAUCinf | 147.8992 | 147.3448 | 99.63 | 96.08 | 103.30 | 82.6704 | 127.6919 | 154.46 | 119.34 | 199.91 |

It can be seen that there is no food effect in case of Test formulation wherein the Pharmacokinetic parameters are relatively unaltered in both the fed and fasted conditions. The Reference formulation shows a much lower absorption profile as compared to the Test formulation in fasted conditions. However, in presence of food, the absorption of the Reference formulation is increased, thereby indicating a food effect.

The formulation and dosage form of the present invention has thus demonstrated unexpectedly enhanced bioavailability, simplicity of design and manufacture and absence of food effect.

Various modifications of the system of the invention may be made without departing from the spirit or scope of the invention. The above description and examples illustrate various aspects of the invention and should not be construed to limit its scope.

1. A fenofibrate formulation with enhanced oral bioavailability comprising fenofibrate dissolved in a lipophilic surfactant.

2. The fenofibrate formulation of claim 1, additionally comprising a hydrophilic surfactant and wherein the lipophilic and hydrophilic surfactants are present in a weight ratio of 1:2 to 2:1.

3. The fenofibrate formulation of claim 2, wherein the lipophilic surfactant is a fatty acid ester of propylene glycol and wherein it is used in amounts of 20% w/w to 80% w/w of the formulation.

4. The fenofibrate formulation of claim 3, wherein the lipophilic surfactant is propylene glycol monocaprylate.

5. The fenofibrate formulation of claim 2, wherein the hydrophilic surfactant is selected from the group of polyoxyethylene-polyoxypropylene block copolymers and polyoxyethylene sorbitan fatty acid esters and wherein it is used in amounts of 80% w/w to 20% w/w of the formulation.

6. The fenofibrate formulation of claim 5, wherein the hydrophilic surfactant is selected from the group of polysorbate 80, poloxamers 108, 188, 217, 238, 288, 338 and 407.

7. The fenofibrate formulation of claim 2, further comprising excipients selected from the group of antioxidants, pH stabilizers, buffers, preservatives, thickeners, colors and flavours.

8. The fenofibrate formulation of claim 2, comprising 30 mg to 200 mg of fenofibrate, 20% w/w to 80% w/w of fatty acid ester of propylene glycol and 80% w/w to 20% w/w of hydrophilic surfactant selected from the group of polyoxyethylene-polyoxypropylene block copolymers and polyoxyethylene sorbitan fatty acid esters.

9. The fenofibrate formulation of claim 2, comprising 40 mg to 160 mg of fenofibrate, 20% w/w to 80% w/w of propylene glycol monocaprylate and 80% w/w to 20% w/w of polysorbate 80.

10. The fenofibrate formulation of claim 2 further comprising one or more additional active agents.

11. The fenofibrate formulation of claim 10 wherein the additional active agents are selected from the group of anti-diabetic agents, cardiovascular agents, nicotinic acid, HMG-CoA reductase inhibitors, sterol absorption inhibitors and bile acid sequestrants.

12. The fenofibrate formulation of claim 10 wherein the additional active agent is atorvastatin and wherein the formulation comprises of 5 mg to 80 mg atorvastatin and 30 mg to 200 mg fenofibrate.

13. A capsule for oral administration comprising the fenofibrate formulation of claim 1.

14. A process for manufacture of fenofibrate formulation with enhanced oral bioavailability comprising dissolving fenofibrate in a lipophilic surfactant, optionally adding a hydrophilic surfactant and mixing to obtain a clear or slight hazy solution.

15. The process of claim 14 comprising dissolving about 0.1% w/w to 50% w/w of fenofibrate in about 20% w/w to 80% w/w of lipophilic surfactant and adding about 80% w/w to 20% w/w of hydrophilic surfactant to obtain a clear or slight hazy solution.

16. The process of claim 14 comprising the following steps:
i. dissolving fenofibrate and optionally a hydrophilic surfactant together or sequentially, in a lipophilic surfactant,
ii. stirring the mixture well and applying heat if necessary, to produce a clear or slight hazy solution,
iii. optionally adding other excipients to the mixture of step b,
iv. incorporating the mixture into a dosage form

17. The process of claim 14 comprising the following steps:
i. dissolving fenofibrate and a hydrophilic surfactant together or sequentially, in a lipophilic surfactant,
ii. stirring the mixture well and applying heat if necessary, to produce a clear or slight hazy solution,
iii. optionally adding other excipients included from the group of antioxidants, pH stabilizers, buffers, preservatives, thickeners, colors and flavours to the mixture of step b,
iv. incorporating the mixture into a capsule dosage form.

18. The process of claim 14 comprising the following steps:
i. dissolving fenofibrate and a hydrophilic surfactant selected from the group of Poloxamers and Polysorbates, together or sequentially in an ester of propylene glycol,
ii. stirring the mixture well and applying heat if necessary, to produce a clear or slight hazy solution,
iii. optionally adding other excipients included from the group of antioxidants, pH stabilizers, buffers, preservatives, thickeners, colors and flavours to the mixture of step b,
iv. incorporating the mixture into a soft gelatin capsule dosage form.

19. The process of claim 14 comprising the following steps:
i. Dissolving fenofibrate, one or more additional active agents and optionally a hydrophilic surfactant, together or sequentially in a lipophilic surfactant,
ii. stirring the mixture well and applying heat if necessary, to produce a clear or slight hazy solution,
iii. optionally adding other excipients to the mixture of step b,
iv. incorporating the formulation into a dosage form.

20. The process of claim 14 comprising the following steps:
i. Dissolving fenofibrate, atorvastatin and a hydrophilic surfactant together or sequentially, in a lipophilic surfactant,
ii. stirring the mixture well and applying heat if necessary, to produce a clear or slight hazy solution.
iii. optionally adding other excipients included from the group of antioxidants, pH stabilizers, buffers, preservatives, thickeners, colors and flavours to the mixture of step b,
iv. incorporating the formulation into a capsule dosage form.

21. The process of claim 20, wherein the hydrophilic surfactant is selected from the group of Poloxamers and Polysorbates, lipophilic surfactant is an ester of propylene glycol and the dosage form is a soft gelatin capsule dosage form.
